(19) United States
(12) Patent Application Publication
(10) Pub. No.: US 2014/0234391 A1
(43) Pub. Date: Aug. 21, 2014

(54) LOCATION-SPECIFIC BACTERIAL MANAGEMENT

(71) Applicant: Kishore Murthy, Orleans (CA)

(72) Inventor: Kishore Murthy, Orleans (CA)

(21) Appl. No.: 14/130,045

(22) PCT Filed: Jun. 29, 2012
(86) PCT No.: PCT/CA2012/050447

§ 371 Date: Apr. 2, 2014

Related U.S. Application Data
(60) Provisional application No. 61/503,388, filed on Jun. 30, 2011.

(51) Int. Cl. A61K 35/76

(52) U.S. Cl. CPC A61K 35/76
USPC 424/438; 424/93.6; 424/451; 424/439

(57) ABSTRACT

A method for reducing the population of pathogenic bacteria in an animal being reared in an animal rearing facility is provided. The method involves using one or more than one naturally-occurring location-specific bacteriophages selected to be highly specific to the strains of the one or more than one pathogen present in the facility, a phage component derived from the one or more than one naturally-occurring location-specific bacteriophages, or a combination thereof, to produce a location-specific bacteriophage preparation. The preparation is administered to the animal, to reduce the one or more than one pathogenic bacteria. The method may also involve a step of identifying location-specific isolates of the one or more than one pathogenic bacteria at or near the animal rearing facility.

### FIELD OF INVENTION

The present invention provides methods to reduce pathogenic bacteria in animal rearing and aquaculture facilities. More specifically, the methods involve the use of bacteriophage isolated from a location that includes the facility and selected to be specific to target pathogenic bacteria in an animal rearing facility.

### BACKGROUND OF THE INVENTION

Contamination of farm animals, aquatic animals and birds by pathogenic bacteria is a major problem for the animal rearing and aquaculture industry. Some of these pathogenic bacteria that cause disease in the animals are zoonotic in nature and are a public health concern. This is further complicated by the fact that there has been an increase in the number of antibiotic resistant bacteria reducing the number of available treatment options. Of particular concern are pathogens such as multidrug resistant Staphylococcus aureus (including MRSA), multidrug resistant Salmonella spp, Escherichia coli, Campylobacter jejuni, Streptococcus spp., Clostridium difficile, Clostridium perfringens, Pseudomonas aeruginosa and others.

Contamination of meat and meat products destined for human consumption is an ongoing problem in the food industry. Of particular concern are Escherichia coli, Salmonella spp., and Campylobacter spp. pathogens, all of which do not cause disease in these animals but can cause food-borne illnesses in humans. Human illnesses due to these pathogens are often caused by the consumption of uncooked or under-cooked contaminated meat products from food animals including chicken, turkey, beef and dairy cattle and swine. In addition pathogens carried in manure from these production animals may be transferred to water sources used for irrigation and lead to contamination of agricultural produce such as lettuce and other leafy greens.

Sources of bacterial contamination for food animals such as pigs, poultry, and cattle are numerous, and include water, unclean bedding and holding areas and feed. Contamination of feed can occur during processing, storage or transportation. Cross contamination of animals with pathogens also occurs at the pen/barn level since they consume feed and water from common sources (feed/water troughs/feed bunks and the like). By example, one common source of contamination of meat and meat products from beef cattle is at the abattoir, where contamination of the carcass could occur from bacteria transferred from the hide or hair during the skinning process, from faeces in the gut during the evisceration process or other procedures during the slaughter process.

Efforts to reduce human food-borne illnesses, typically involve post-harvest interventions at the processing plant level rather than at the pre-harvest stage. An example of a pre-harvest method to reduce food-borne illness involves the use of antibiotics to treat animal disease and reduce contamination of the herds/flocks with bacterial pathogens (e.g. Ransom et al (2003) Research Fact Sheet, National Cattleman's Beef Association, Centennial Colo.; Dunn et al (2004) J Food Prot 67, 2391-2396; Kuhnert et al (2005) Vet Microbiol, 109, 37-45). Extensive use of antibiotics is known to contribute to the development of bacterial resistance to the antibiotic used. A European Union ban on the use of antibiotics as growth promoters in animal has reduced its use for this purpose in the EU. Reducing the use of antibiotics for growth promotion purposes will help reduce the widespread dissemination of antibiotic resistance.

Use of antibiotics is the most common therapeutic intervention for the treatment of bacterial diseases in animals, both food animals and other farm and aquatic animals, as well as pets such as dogs and cats. It is also widely used in aquaculture. Many of these pathogens causing disease in these animals are becoming resistant to multiple antibiotics and require new approaches for treatment.

U.S. Pat. No. 5,965,128 (see also Zhao et al (1998) J Clin Microbiol, 36, 641-647) teach the use of three different strains of E. coli found in the cattle gut as probiotic bacteria to reduce or prevent the carriage of E. coli O157:H7. This method involves inoculation of cattle via rumen cannulation which is not practical for administering treatment in a commercial setting. Brashears et al (2003, J Food Prot, 66, 748-754), and Younts-Dahl et al (2004, J Food Prot, 67, 889-893) describe that supplementation with Lactobacillus, Propionibacterium microbials, or both, can decrease E. coli O157:H7 shedding in cattle. However, this treatment does not eliminate the pathogen. Similar results were obtained by Garner and Ware (US 2003/0175305, US 2003/0175306, US 2003/0175307, and WO 2004/030624).

Having an antibacterial treatment that is targeted to the pathogen of interest, does not alter the gut microflora and also does not alter the farm's environment of beneficial microbes is desired.

Bacteriophages (or “phages”) are bacterial viruses that specifically infect and kill bacteria and are widely distributed in nature having been identified in all environmental compartments; in soil, water bodies, animals, plants etc. and are the most abundant organism on earth (Hendrix et. al, PNAS USA, 96-2192-2197, 1999). Phages recognize receptors on the bacterial surface, attach to them and inject their genetic material into the host cell. They degrade the host bacteria's DNA and synthesize their own genetic material and required coat proteins, re-assemble multiple copies of bacteriophage particles before bursting the cell. The released bacteriophages infect and destroy additional bacteria in the surrounding environment. This process continues until all the bacteria are eliminated from the system. Bacteriophage use to reduce pathogen load in food animals has been evaluated in studies by different groups.

U.S. Pat. No. 6,656,463 discloses reduction of Salmonella populations within swine using Felix 0-1 phage. Similarly, Smith et al (J Gen Microbiol (1987) 133, 1111-1126) have shown that phages may be used to control enterotoxigenic E. coli infection in livestock. However, the phage were efficient when administered prior to or simultaneously with E. coli administration. Phages used in this study were highly specific to enterotoxigenic E. coli and are not useful in reducing food-borne illness caused by E. coli O157:H7 since they do not recognize this pathogen.

Kudva et al (Appl Env Microbiol (1999) 65, 3767-3773) showed that a mixture of three E. coli O157-specific phage were effective in reducing the amount, or clearing, of E. coli O157:H7 from cultures in vitro. In other similar studies, phages have been shown to be effective in in vitro studies, but no or low, effectiveness was observed when these phages were tested in vivo (see Callaway T. R. et al. (2004) J Animal Sci. 82 (E. Suppl):E93-99 for review). Further improvements are needed so that bacteriophage are effective in reducing E. coli O157:H7 infection of cattle.

U.S. Pat. No. 6,485,902 (Waddell et al) teaches the use of specific bacteriophages to reduce the levels of E. coli O157:H7 in the gastrointestinal tract of cattle. A mixture of six phages was administered orally along with milk and at high dosages to calves prior to and after challenge with E. coli O157:H7. A reduction in shedding of E. coli O157:H7 was observed in feces compared to calves not receiving phages. Phages used in this study were from a collection of E. coli O157:H7 typing phages.

To date, bacteriophage-based treatments being developed are aimed at the use of bacteriophages that have a very broad host range capable of acting on a majority of the isolates of the pathogen found around the globe. For this, bacteriophages from different geographic regions, sometimes from different continents, have to be included in the cocktail. Even though phage cocktails that are effective against most of the isolates of a given target pathogen can be developed, their effectiveness varies significantly between isolates from different regions. Developing a location-specific approach tailored to facilities in a given area will improve treatment efficacy and have minimal impact on the local bio-environment.

### SUMMARY OF THE INVENTION

The present invention provides methods to reduce pathogenic bacteria in animal rearing and aquaculture facilities. More specifically, the methods involve the use of bacteriophage isolated from a location that includes the facility and selected to be specific to target pathogenic bacteria in an animal rearing and aquaculture facility.

The present invention provides methods to control pathogenic bacteria in an animal, animal rearing facility, animal production systems such as a feedlot, rearing enclosure, an aquaculture facility and the like, or a combination thereof, preferably using bacteriophages found in the same environment or the same geographic region, phage parts derived from these phages or a combination thereof.

The invention presented herein uses phage obtained from specific geographic regions. Bacteriophages isolated from the same region as that of the animal rearing facilities are found to be most effective on target bacterial isolates from that region. With this method, phages isolated from a region of interest are added back to the same region and have minimal impact on the local bio-environment.

It is an object of the present invention to provide an improved method of location-specific bacterial management.

The present invention provides a method for reducing bacteria within an animal being reared in an animal rearing facility using bacteriophages, bacteriophage components or a combination thereof that are specific to the strains of the pathogen in that facility and that have preferably been isolated from a location of the facility. Bacteriophages that are specific to the pathogen in question, but found outside the facility but in the same location as the animal rearing facility may also be used when necessary. In addition, other bacteriophage obtained from one or more collections may be used to supplement the location-specific bacteriophage. The method of reducing pathogenic bacteria within one or more than one animal being reared in an animal rearing facility involves

a. identifying one or more than one pathogenic bacteria at a location of the animal rearing facility;

b. isolating one or more than one bacteriophage strain from the animal rearing facility or from a location of the facility that exhibits antibacterial activity against the one or more pathogenic bacteria to obtain a location-specific bacteriophage preparation, the location-specific bacteriophage preparation comprising the one or more than one bacteriophage strain, phage components obtained from the one or more than one bacteriophage strain, or a combination thereof;

c. inoculating the one or more than one animal with the location-specific bacteriophage preparation, thereby reducing the pathogenic bacteria within the one or more than one animal being reared in the animal rearing facility.

The method as defined above may also include an additional step (d) of repeating steps (a) to (c) after a period of time to reduce any additional pathogenic bacteria that may be identified. For example, the period of time may include a time interval of 1 month to 48 months or any time interval therebetween.

The one or more than one pathogenic bacteria may be selected from the group Escherichia coli, Streptococci, Humicola, Salmonella, Campylobacter, Listeria, Lawsonia, Staphylococcus, Pasteurella, Mannheimia, Mycobacterium, Hemophilius, Helicobacter, Mycobacterium, Mycoplasmi, Nesseria, Klebsiella, Enterobacter, Proteus, Bactercides, Pseudomonas, Borrelius, Citrobacter, Propionobacter, Treponema, Shigella, Clostridium, Enterococcus, Leptospirex, Bacilli including Bacillus anthracis, Aeromonas, Renibacterium, Edwardsiella, and Vibrio

If desired, additional bacteriophage strains may be added to the location-specific bacteriophage preparation prior to the step of inoculating. The additional bacteriophage strains may be obtained from sources that were not obtained from the location where the animal rearing facility is located. Examples of non-regional sources may include, public bacteriophage collections, veterinary labs, diagnostic labs, depositories (e.g. the ATCC), or bacteriophages isolated from other areas in the country or adjoining countries. Effectiveness of the bacteriophage preparation on the target pathogenic bacteria in the facility should be verified prior to its use.

The present invention provides a method for reducing a population of one, or more than one pathogenic bacteria present in an animal at an animal rearing facility comprising, administering one or more than one bacteriophage strain isolated from the animal rearing facility or from the location of the facility, phage components derived from the phage, or a combination thereof, to the animal, the one or more than one bacteriophage strain, phage components, or a combination thereof, acts on the one or more than one pathogenic bacteria and reduces the population of the one or more than one pathogenic bacteria in the animal.

Identifying and using bacteriophages from at a location of the facility where treatment for a particular pathogen is required has many advantages including: (i) highly effective treatment tailored to the animal rearing facility, (ii) minimal change in bio-environment since no new chemicals or organisms are added to the local environment, (iii) treatment can be easily adapted to new variants of the pathogen in the region since the treatment is tailored to the facility, (iv) no possibility of development of organisms resistant to antibiotics used in human medicine, (v) can be used along with other therapeutic treatments with no adverse effects, to name a few.

In the methods described above, target pathogenic bacteria may be first isolated from the animals by taking swabs from the infected areas or from manure using standard microbiology protocols, for example, J. Howard and D. M. Whitcombe. (1995, Diagnostic Bacteriology Protocols which is incorporated herein by reference). These target pathogenic bacterial isolates are characterized to confirm their identity and further analyzed to determine their strains as well as serotypes. Prevalence data for each of the serotypes of the target pathogenic bacteria in the animal rearing facility may also be collected and the most prevalent serotypes of the bacterial isolates are used for development of a bacteriophage-based treatment.

Bacteriophages highly specific to the target pathogenic bacterial isolates may be present in a few carrier animals, in different locations in and around the rearing facility and surrounding areas, or both. For bacteriophage isolation, samples are collected from several areas in the rearing facility including swabs from animals, manure, water from water troughs and other water bodies in the facility, bedding, animal handling machinery and tools and other areas in the facility. Soil and water samples from different areas in the vicinity of the animal rearing facility are also collected and used for bacteriophage isolation. Similar samples are collected from other facilities in a location where the farm is located and used for bacteriophage isolation.

Bacteriophages may be isolated from samples obtained as outlined above using standard phage isolation protocols (Molecular cloning: a laboratory manual, Sambrook et al., 1989) to produce a location-specific bacteriophage preparation. Isolated bacteriophages are characterized using standard techniques (Molecular cloning: a laboratory manual, Sambrook et al., 1989) and categorized based on their in-vitro efficacy, including their host range on bacterial isolates of interest as well as their plating efficiency etc. on the most prevalent isolates of the target pathogen in the facility. Phages that show broad host range, are distinct from each other at the molecular level as determined by techniques such as RFLP using multiple enzymes and other techniques and show a good safety profile including lack of virulence factors and known toxins, show lack of transduction potential and other properties are developed further. These phages may also be used for developing phage components to be used for treatment purposes.

A location-specific bacteriophage preparation comprising phages and phage components may be used in one of the following formats: as a liquid without any further processing, stabilized in liquid form, immobilized and lyophilized, encapsulated, provided in tablet form, provided in capsule form, immobilized onto a solid support or a combination of the above forms. To make it convenient for application to wounds and other surface applications, phages and phage components in any of the above forms may be admixed in cream, lotion, gel or lubricant or a combination thereof.

Bacteriophage or phage components described above may also be administered by adding to animal feed or drinking water, by inhalation, or injection either intramuscular, intraperitonial, or intrathecal, or by administering rectally, topically, or a combination of these methods.

A location-specific bacteriophage preparation may also be admixed with pelleted feed for administration to production animals such as poultry and swine, pets such as dogs and cats and the like. For example, the location-specific bacteriophage preparation may be immobilized, or lyophilized and mixed with pelleted feed immediately after the pelleting process. As an example, bacteriophages immobilized covalently onto a solid support using technology as outlined in U.S. Pat. No. 7,482,115 (Scott Hugh and Michael Mattey, 2009) may be mixed-in with liquid binders such as molasses, desugared molasses, sugar syrup, corn steep liquor, condensed liquid whey, edible oil, wax, edible polymers, gums, vegetable gums, cellulose, and other liquid binders and applied to the pellets as they are being extruded from the pelletizer and cooled to temperatures below 50° C. This process can be easily achieved by minor modification to pelleting machines that are currently available on the market. These bacteriophage containing pellets can be mixed-in with regular pelleted feed at a defined ratio for administration to the animals.

Use of bacteriophages for treating bacterial contamination in food animals to improve the safety of meat and meat products has been described earlier. See for example, WO2006/047870; WO2006/047871; WO2006/047872 and WO2006/125319. Bacteriophages can be stabilized by adsorption onto a solid support (WO2006/047870; which is incorporated herein by reference) and subsequently immobilized using different encapsulation media as described in WO2006/047871; WO2006/047872 (which are incorporated herein by reference). The application of these encapsulated phages to treat animals in large animal holding facilities is described in WO2006/125319 (which is re incorporated herein by reference). Bacteriophages can also be used for treating pathogens in animal manure prior to spreading them on fields (WO2006/125318; which is incorporated herein by reference). All the above applications deal with the use of bacteriophages obtained from global cultures or that are used in a global setting. The bacteriophages used for the treatments described in these references are not location-specific, or restricted to those isolated from a given location. Phages for these applications are selected so as to have global host range and may or may not be location-specific or found at the treatment location. Furthermore, the treatment for isolates of the target pathogen in a given facility is not evaluated prior to the treatment.

Using one of the processes described above, one or more than one bacteriophage, phage component or combination thereof, may be administered in a treatment dosage of about 10³ to about 10¹³ pfu per animal per treatment from about 1 to about 15 days. Alternatively, the one, or more than one bacteriophage strain, or phage components, may be administered in a maintenance dosage of about 10² to about 10¹⁰ pfu per animal per treatment for the next 10 to 90 days. In yet another alternative, the one or more than one controlled release bacteriophage strain, or phage components, may initially be administered in a treatment dosage of about 10³ to about 10¹³ pfu per animal per treatment from about 1 to about 15 days, followed by a maintenance dosage of about 10² to about 10¹⁰ pfu per animal per treatment for the next 10 to 90 days. The number of treatments given to the animal may vary from 1 to 3 per day depending on the indication being treated.

The use of bacteriophages, phage components or a combination thereof for reducing pathogens in the gut of food animals helps improve the safety of food sources as well as help reduce pathogen contamination of agricultural produce, source water, pets, and the environment in general. Target pathogen specific bacteriophage, phage components or both, can be safely administered to animals without affecting the nonpathogenic bacterial flora naturally present in the animal or the environment.

The process using bacteriophages that have been isolated from an animal rearing facility or its vicinity for use in treating one or more desired pathogens in the same facility overcomes several disadvantages of prior art. By using this tailor-made approach, the efficacy of the bacteriophage preparation is very high as compared to phages from a global collection. This includes a higher plating efficiency (EOP) on target pathogens found in the facility making them more efficacious in treating the disease, a broader host range on the isolates found in the facility since phages in the preparation have been selected to be specific to the isolates of the target pathogen(s) that are present in the rearing facility, or both a higher EOP and a broader host range when compared to a reference or standard bacteriophage population. For example, the efficiency of plating (EOP) of the phage on isolates of the location-specific target pathogen may be compared to the EOP of a standard strain of bacteriophage obtained from an established collection such as American Type Culture Collection (ATCC) or other collections including global phage collections. Location specific phages, producing titers in the range of 10 to 100,000 times higher than those obtained with a standard strain of bacteriophage or phages obtained from a global collection, when plated on the bacterial isolate of interest, may be considered as having a higher EOP. Phages that target one or more additional bacterial isolates found in the facility when compared to the number of isolates targeted by a standard strain of bacteriophage obtained from established collections, may be considered as having a broader host range

Furthermore, the methods provided herein are all-natural, using phages obtained from the same location or from the region where the treatment is applied and assures that no extraneous chemicals are introduced into the facility's environment unlike antibiotics or other chemical antibacterials. This approach limits the widespread use of bacteriophages from other regions. However, additional bacteriophage isolated from other regions but showing specificity to the target pathogen in the location of interest may be used to supplement the location-specific bacteriophage preparation if necessary.

Target bacterial isolates change over time producing new bacterial variants. Phages isolated and selected for inclusion in the phage bank for the region can be tested against new variants as they appear and sub populations of phages that efficiently plate on these new variants identified. Since phages evolve along with bacterial isolates, a subpopulation of phages, effective against the new variants of the pathogen may also be identified in the same facility or in a phage bank prepared for that region. These phages can be used for treating animals in that facility that are carrying the variant strain of the bacterial pathogen.

Bacteria isolated from the different facilities may be maintained in a regional bacterial collection. This collection is updated on an annual basis to determine if any new variants have developed. Effectiveness of phages in a regional phage bank is tested on the new additions to the bacterial collection to determine if any additional phages need to be isolated and added to the bacteriophage bank to have effective coverage of all target pathogenic bacteria in the location. The regional bacterial collection may initially be updated on an annual basis with the frequency of further updates determined by the results of updates in the first 2-3 years.

Resistance development against phages can be minimized by using two or more phages for treatment, with each of the phages directed against different receptors on the bacterial surface This can be constantly updated using any one of the following methods: using other characterized phages that are already present in the regional bacteriophage bank, isolating new phages from the facility, or supplementing the location-specific bacteriophage preparation with additional bacteriophages obtained from other regional collections. The high degree of control and specificity introduced by the methods described herein reduce the possibility of development of widespread resistance (unlike what has been seen with the development of antibiotic resistance) and have minimal negative impact on the environment.

This summary of the invention does not necessarily describe all features of the invention.

### DESCRIPTION OF PREFERRED EMBODIMENT

The present invention provides methods to reduce pathogenic bacteria in animal rearing and aquaculture facilities. More specifically, the methods involve the use of bacteriophage isolated from a location that includes the facility and selected to be specific to target pathogenic bacteria in an animal rearing and aquaculture facility.

The following description is of a preferred embodiment.

The present invention relates to methods for reducing pathogenic bacteria within an animal and an animal rearing facility using antibacterial agents specific to the pathogen and found in the same environment. More specifically, methods are provided to control pathogenic bacteria in an animal, animal rearing facility, animal production systems such as a feedlot, rearing enclosure, and the like, or a combination thereof, preferably using location-specific bacteriophages found in the same environment including geographic region where the facility is located, phage parts derived from these phages or a combination thereof.

The present invention provides a method for reducing a population of one, or more than one target pathogen present in an animal, comprising, administering one or more than one bacteriophage strain that has been isolated for the same environment or location (i.e. location specific) as the bacteria, phage components derived from these phages or a combination thereof to the animal, such that the one, or more than one bacteriophage strain, or phage components, acts to clear the one or more than one pathogen from the animal.

By “location-specific” or “location” as used herein, it is meant a location that includes the facility in which bacteria are being treated, or in some cases the location may be in the area of the facility, for example adjacent to the facility. The size of a location-specific area may be a site, a local area, a regional area, or a continent. By “site” it is meant that this location is about 0 to about 10 km or any distance therebetween from the facility, for example 1, 2, 4, 6, 8, 10 km or any distance therebetween, from the facility. The site area may also be located adjacent to the facility. By “local area” it is meant a location that is from about 11 to about 500 km or any distance therebetween, for example 11, 15, 20, 25, 30, 35, 40, 45, 50, 75, 100, 125, 150, 175, 200, 250, 300, 350, 400, 450, 500 km or any distance therebetween, from the facility. By “regional” it is meant a location is from about 501 to about 3,000 km or any distance therebetween, for example 501, 600, 700, 800, 900, 1,000, 1,250, 1,500, 1,750, 2000, 2,250, 2500, 2750, 3000 km or any distance therebetween, from the facility. By “continental” it is meant one of the know continents for example, The Americas, Asia, Africa, Australia, and Europe.

The method of reducing pathogenic bacteria within one or more than one animal being reared in an animal rearing facility involves, identifying one or more than one location-specific pathogenic bacteria, for example obtained at or near the animal rearing facility, for example at a site, local area, region or within the same continent as the facility, isolating one or more than one location-specific bacteriophage strain from or near the animal rearing facility or in the location of the facility, that exhibits antibacterial activity against the one or more pathogenic bacteria to obtain a location-specific bacteriophage preparation, the location-specific bacteriophage preparation comprising the one or more than one bacteriophage strain, phage components obtained from the one or more than one bacteriophage strain, or a combination thereof, and inoculating the one or more than one animal with the location-specific bacteriophage preparation, thereby reducing the pathogenic bacteria within the one or more than one animal being reared in the animal rearing facility. The method may also include an additional step of repeating the steps of identifying, isolating and inoculating as described above, after a period of time, to reduce any additional pathogenic bacterial isolates of the target bacteria that may be identified. For example, the period of time may include a time interval of 1 month to 48 months or any time interval therebetween.

Also provided is a method for reducing a population of one, or more than one target pathogen present within an animal rearing facility comprising, providing one or more than one location-specific bacteriophage strain isolated from the geographic region of the facility, for example a site, local area, region, or within the same continent as the facility, or phage components derived from the bacteriophage strains isolated from the location, site, local area, region, or within the same continent as the facility, to animal feed, drinking water, an animal, or a combination thereof, such that the one or more than one bacteriophage strain, phage component, or both, adsorb to the target pathogen thereby kills the pathogen and reduces the population of the one or more than one target pathogen within the animal or the animal rearing facility. The animal rearing facilities may include, but are not limited to a poultry farm, large and small animal breeder farms, a hatchery, a pig nursery, a grow-finish operation, a holding pen for beef cattle, a rearing enclosure, including for example a rearing barn or rearing pen, a petting zoo, a horse stable, other animal housing quarters, an aquaculture facility and the like.

By the term “target pathogen” or “target bacteria”, it is meant pathogenic bacteria that may cause illness in humans, animals, fish, birds, or plants. The target pathogen may be any type of bacteria, for example but not limited to, E. coli, Streptococci, Humicola, Salmonella, Campylobacter, Listeria, Staphylococcus, Pasteurella, Mannheimia, Mycobacterium, Hemophilius, Helicobacter, Mycobacterium, Mycoplasmi, Nesseria, Klebsiella, Enterobacter, Proteus, Bactercides, Pseudomonas, Borrelius, Citrobacter, Propionobacter, Treponema, Shigella, Clostridium, Enterococcus, Leptospirex, Bacilli including Bacillus anthracis, Aeromonas, Renibacterium, Edwardsiella, Vibrio and other bacteria pathogenic to humans, animals, fish, birds, or plants. Target bacteria are isolated from several animals and different parts of the rearing facility and characterized in detail to determine the most prevalent isolates of the target pathogen. These isolates are used to create a select panel of target bacteria to be used for isolation of phages suitable for treatment at that facility.

By the term “animal” or “animals”, it is meant any animal that may be affected by, or carry, a pathogen. For example, but without wishing to be limiting in any manner, animals may include poultry, such as chicken or turkey, etc; swine; livestock, which term includes all hoofed animals such a horses, cattle, goats, and sheep, etc; other domesticated animals and household pets such as cats and dogs; and aquatic animals such as fish, shrimp, crab etc.

The term “bacteriophages” or “phages” is well known in the art and generally indicates a virus that infects bacteria. Phages are parasites that multiply inside bacterial cells by using some or all of the host's biosynthetic machinery, and can either be lytic or lysogenic. The bacteriophages used in accordance with the present invention may be any bacteriophage, lytic or lysogenic that is effective against a target pathogen of interest. However, the bacteriophages for use in the present invention are preferably selected to be non-lysogenic, which means that the phage DNA is not incorporated into the host's genomic DNA following phage infection, and have been isolated from the environment of the facility where the treatment is to be applied. Phage specific for one or more than one target pathogen may be isolated using standard techniques known in the art for example as taught in Sambrook et al (1989, Molecular cloning: a laboratory manual, Cold Spring Harbor Laboratory, Cold Spring Harbor, N.Y.; which is incorporated herein by reference). If desired, a cocktail of different bacteriophage may be used to target one or more than one pathogen as described herein.

Similarly, “phage component” or “phage components” may comprise any phage component including but not limited to the tail, or a phage protein that is effective in killing, reducing growth, or reproduction of a target bacteria, or a plurality of target bacteria.

If desired, a cocktail of bacteriophages, phage components, or both, may be used against a single bacterial target, or multiple bacterial targets. The target bacteria may be any type of bacteria, for example but not limited to the bacterial species and strains of Escherichia coli, Streptococci, Humicola, Salmonella, Campylobacter, Listeria, Lawsonia, Staphylococcus, Pasteurella, Mannheimia, Mycobacterium, Hemophilius, Helicobacter, Mycobacterium, Mycoplasmi, Nesseria, Klebsiella, Enterobacter, Proteus, Bactercides, Pseudomonas, Borrelius, Citrobacter, Propionobacter, Treponema, Shigella, Clostridium, Enterococcus, Leptospirex, Bacilli including Bacillus anthracis, Aeromonas, Renibacterium, Edwardsiella, Vibrio and other bacteria pathogenic to humans, animals, fish, birds, or plants. Of interest are bacteria that are known to contaminate animal feeds, liquid animal feeds, animal feedlots, animal holding facilities, animal kennels, aquatic enclosures, as well as pathogens causing disease in production animals, and other domesticated animals. Of particular interest in the food safety sector are bacteria that infect livestock, including swine, cattle and poultry destined for human consumption for example but not limited to Salmonella, Campylobacter and E. coli O157:H7. In the case of bacteria causing disease in production and domesticated animals for example but not limited to Escherichia coli, Clostridium, Streptococcus, Staphylococcus, Pasteurella, Mannheimia, Treponema, Pseudomonas, Aeromonas, Renibacterium, Edwardsiella, Vibrio and others

The bacteriophages, or phage components, may be provided in an aqueous solution. The aqueous solution may be any solution suitable for the purpose of the present invention. For example, the bacteriophages, or phage components, may be provided in water or in an appropriate medium as known in the art, for example LB broth, SM, TM, PBS, TBS or other common buffers. For example, but without wishing to be limiting, the bacteriophages may be stored in LB broth.

The bacteriophages or phage components also may be provided in a dry form for admixing with either a liquid animal feed or an animal feed. Having phages or phage components in these formats helps improve their stability and storage characteristics. Examples of dry forms of bacteriophages or phage components include but are not limited to lyophilized bacteriophages or phage components, bacteriophages or phage components that are immobilized on a matrix, bacteriophages or phage components that are encapsulated, bacteriophages or phage components that are provided in capsule form, bacteriophages or phage components that are provided in tablet form, or a combination thereof, for example as described in WO 2006047870, WO 2006047871, WO 2006047872, WO 200612531, WO 2006125318 (which are incorporated herein by reference).

For certain other applications such as treatment of wounds and other surface applications, the bacteriophages or phage components provided in any of the above formats may be mixed-in with cream, lotion, gel or lubricant or a combination thereof (for example as described in WO 2006047870, which is incorporated herein by reference).

The bacteriophage compositions of the present invention may be mixed with the feed of livestock, birds, poultry, domestic animals and fish, to aid in reducing the shedding of target bacteria or to cure an infection in these animals. Bacteriophages or phage components, present as a liquid, immobilized, encapsulated, capsulated, tablet or a combination thereof, may be mixed with other additives or supplements applied to animal feed, as part of the daily feed regime, as needed or incorporated into pelleted feed, thus, settling of the bacteriophages, or phage components, in the feed could be avoided. The bacteriophage or phage components may also be admixed with drinking water. Additionally, alternate forms of administration, for example but not limited to inhalation, injection, intraperitonial, intramuscular, intrathecal, vaginal, rectal, topical or a combination thereof, may be used to administer the bacteriophages, phage components, or both of the present invention.

Lyophilization of bacteriophage or phage components can be carried out using any known lyophilization procedure, for example but not limited to methods disclosed in Clark and Geary (1973, Preservation of bacteriophages by freezing and freeze-drying, Cryobiology, 10, 351-360; Ackermann et al. 2004, Long term bacteriophage preservation, World Federation Culture Collections Newsletter, 38, 35 (which are both incorporated herein by reference).

The bacteriophages, or phage components, may also be provided immobilized onto a matrix, either covalently immobilized, for example, as described in U.S. Pat. No. 7,482,115 (which is incorporated herein by reference) onto the matrix, or non-covalently immobilized for example as described in WO 2006/047870 (which is incorporated herein by reference) onto the matrix. By the term “matrix”, it is meant any suitable solid matrix that is either soluble in water, ingestible by an animal, or suitable for use with solid or liquid animal feed. For certain applications, the matrix may be non-water-soluble, and any absorbed phages can be released from the matrix within an aqueous environment. For such applications the matrix should be capable of adsorbing the bacteriophages, or phage components, onto its surface and releasing the bacteriophages, or phage components, in an appropriate environment. The bacteriophages, or phage components, should not adhere so strongly to the matrix that they cannot be released upon appropriate re-suspension in a medium. Preferably, the adsorbed, immobilized, bacteriophages, or phage components, are non covalently associated with the matrix so that they may be released from the matrix when desired. Non-limiting examples of a matrix that may be used according to the present invention include skim milk powder, soya protein powder, albumin powder, single cell proteins, trehalose, mannitol or other powdered sugar or sugar alcohol, charcoal, latex beads or other inert surfaces, water-soluble carbohydrate-based materials, or a combination thereof. Preferably, the matrix is generally regarded as safe (GRAS). For certain other applications, phage immobilized covalently to the matrix may also be used. These applications include but are not limited to administration of phage in feed or water as well as application to surface wounds, other surface applications and the like. In these applications, upon infection of the pathogenic bacteria by the covalently immobilized phage, new progeny phage are released into the media increasing the local phage concentration which in turn infects additional bacterial pathogens and thus effectively treat the infection. Matrix surfaces such as nylon, other polymers, cellulose etc. may be used for this purpose.

For non-covalent immobilization, the bacteriophages, or phage components, in aqueous solution may be applied to the matrix by any method known in the art, for example dripping or spraying, provided that the amount of the matrix exceeds the amount of aqueous bacteriophage, or phage components, solution. It is preferred that the matrix remain in a dry or semi-dry state, and that a liquid suspension of bacteriophages (or phage components) and matrix is not formed. Of these methods, spraying the bacteriophage solution over the matrix is preferred.

Covalent immobilization of bacteriophages to a solid surface may be carried out using any substance known in the art and any technology known in the art, for example but not limited to immobilization of bacteriophages onto polymeric beads using technology as outlined in U.S. Pat. No. 7,482,115 (which is incorporated herein by reference). Phages may be immobilized onto appropriately sized polymeric beads so that the coated beads may be added to aerosols, creams, gels or liquids. The size of the polymeric beads may be from about 0.1 μm to 500 μm, or any size therebetween in diameter, for example 50 μm to 100 μm or any size therebetween. The coated polymeric beads may be incorporated into animal feed, including pelleted feed and feed in any other format, incorporated into any other edible devise used to present phage to the animals, added to water offered to animals in a bowl, presented to animals through water feeding systems, used for applications such as treatment of surface wounds and other surface treatments and the like using creams, gels, aerosol sprays and the like.

The antibacterial composition comprising immobilized bacteriophages, or phage components, and matrix may be dried at a temperature from about 0° C. to about 55° C. or any amount there between, for example at a temperature of 0, 2, 4, 6, 8, 10, 12, 14, 16, 18, 20, 22, 24, 26, 28, 30, 32, 34, 36, 38, 40, 42, 44, 46, 48, 50, 52, 54 or 55° C. or any amount there between. The antibacterial composition may be dried at a temperature from about 10° C. to about 30° C., or any amount there between, or from about 15° C. to about 25° C. or any amount there between. The drying process may also be accelerated by providing a flow of air over or through the antibacterial composition. Alternatively, drying may be performed by heating the immobilized material under vacuum.

After a period of drying, additional aqueous solution may be applied to the matrix if desired, and the matrix re-dried. This process may be repeated as required to obtain the desired amount of phage on the matrix. The titer of phage on the matrix can be readily determined using standard techniques.

The immobilized or lyophilized bacteriophages, or phage components, may also be encapsulated prior to administration to an animal as a feed additive. By “encapsulated”, it is meant that the immobilized phages, or phage components, are coated with a substance that increases the phages' resistance to the physico-chemical stresses of its environment. The immobilized phages, or phage components, may be coated with any substance known in the art, by any suitable method known in the art, for example, but not limited to US publication 2003/0109025 (Durand et al., which is incorporated herein by reference). In this method, micro-drops of the coating substance are injected into a chamber containing one, or more than one immobilized bacteriophage strain, or phage components, of the present invention and rapidly cooled. Alternatively, a coating composition may be admixed with the one, or more than one immobilized bacteriophage, or phage components, of the present invention, with constant stirring or agitation, and cooled or dried as required.

The coating substance may be any suitable coating substance known in the art. Non-limiting examples of such substances include vegetable fatty acids, fatty acids such as palmitic acid and stearic acid, for example Stearine™, animal waxes, vegetable waxes, for example Carnauba wax and wax derivatives. Other additive molecules may be added to the coating substance; such additive may include antioxidants, sugars, proteins or other synthetic material.

Additional coating substances may also be used, for example, non lipid-based materials (see for example, U.S. Pat. Nos. 6,723,358; and 4,230,687, which are incorporated herein by reference), for example sugars or other carbohydrate-based components that are water soluble. The bacteriophage, or phage component, in the composition of the present invention may also be coated with other substances. Other additive molecules may be added to the coating substance; such additives may include antioxidants, sugars, proteins or synthetic materials.

Several materials for encapsulating the bacteriophages or phage components may be used so that if desired, there is selected release within an animal's gut, release within fermenting liquid feed, release at the location of the wound, while at the same time protecting the bacteriophages, phage components. In addition, bacteriophage or phage components that are encapsulated using non lipid-based materials dissolve in water, releasing bacteriophages or phage components immediately, or soon after mixing with the liquid feed medium. The bacteriophage or phage components may also be released in a time-controlled fashion depending upon the formulation selected, or whether the preparations are provided within a capsule or tablet form. The capsule or tablet formulations may assist in the timed release of the bacteriophage or phage components within the liquid feed medium. Therefore, mixtures of bacteriophages, phage components, or both that are admixed or encapsulated with different materials may be combined and mixed with animal feed, liquid animal feed, or otherwise administered to an animal.

The immobilized or lyophilized bacteriophages, or phage components, may also be provided in a capsule form. By “capsule form”, it is meant that the immobilized phages, or phage components, are provided in a capsule for example a soft capsule, that may be solubilized within an aqueous environment. The capsule may be made of any suitable substance known in the art, for example, but not limited to gelatin, shellac, wax, synthetic or other compounds.

The immobilized or lyophilized bacteriophages, or phage components, may also be provided in a tablet form. By “tablet form”, it is meant that the immobilized phages, or phage components, are provided in a pressed tablet that dissolves in an aqueous environment. The tablet may be made of any suitable substance known in the art, by any suitable method known in the art. For example, the tablet may comprise binders and other components necessary in the production of a tablet as are known to one of skill in the art. The tablet may be an immediate release tablet, where the bacteriophages or phage components are released into the liquid feed upon dissolution of the tablet, or may comprise a timed-release composition, where the bacteriophages or phage components are released within an aqueous environment, including the liquid feed, animal gut, or both in a time-dependent manner. See WO 02/45695; U.S. Pat. No. 4,601,894; U.S. Pat. No. 4,687,757, U.S. Pat. No. 4,680,323, U.S. Pat. No. 4,994,276, U.S. Pat. No. 3,538,214, US (which are incorporated herein by reference) for several examples of time-release formulations that may be used to assist in the time controlled release of bacteriophage, or phage components within aqueous environments.

The immobilized or lyophilized bacteriophage or phage components may be applied onto pelleted feed used for food production animals such as poultry, cattle, swine and sheep, pet animals such as dogs and cats, aquatic animals and the like. Since temperatures generated during the standard pelleting process affect the properties of phage negatively, the immobilized or lyophilized phage preparation can be applied to the pellets immediately after the pelleting process. The phage preparations can be mixed-in with liquid binders such as but not limited to molasses, desugared molasses, sugar syrup, corn steep liquor, condensed liquid whey, edible oil, wax, edible polymers, gums, vegetable gums, cellulose and other liquid binders and applied to the pellets as they are being extruded from the pelletizer and cooled to temperatures below 50° C. This helps generate a bacteriophage containing feed which can be mixed-in with regular pelleted feed at a defined ratio for administration to the animals.

The antibacterial composition of the present invention, in a liquid form, a dry form, including bacteriophages or phage components prepared as described in this invention that are lyophilized or adsorbed onto a matrix, covalently immobilized onto a matrix, encapsulated, or within a capsule or tablet form, or a combination thereof, may be mixed with an animal feed, or a liquid animal feed to produce a treated animal feed, or a treated liquid animal feed, and helps reduce the amount of bacteria in the feed. This treated feed, in either liquid or solid form, may be used to feed any livestock, including cattle, swine, sheep or poultry. The use of controlled release bacteriophages, phage components, or a combination thereof, aids in preventing and treating the bacterial disease affecting the animal or ridding the animal of pathogenic bacteria present in the gut but not causing any disease in the animal, prior to further processing of the animal.

A treated animal feed is an animal feed admixed with an effective amount of an antibacterial composition having one or more than one strain of bacteriophage prepared as described in this invention, one or more phage components from one or more than one strain of bacteriophage, or a combination thereof. The animal feed may be mixed with either a dry or a liquid form of the antibacterial composition. The treated animal feed, or treated liquid animal feed, comprises an effective amount of an antibacterial composition. The treated animal feed may be prepared by any method known in the art. For example, the antibacterial composition may be admixed with the animal feed in a dry form, for example but not limited to, a powder, or a lyophilized preparation may be admixed with the animal feed, or the antibacterial composition may be applied to the animal feed in a liquid form, for example, as a spray, drench, or drip, to produce a treated animal feed. The treated animal feed may then be dried. The effective amount of antibacterial composition having one or more than one strain of bacteriophage, one or more phage components from one or more than one strain of bacteriophage, or a combination thereof, is from about 10² pfu/g to about 10¹³ pfu/g dry wt of animal feed; for example, from about 10³ pfu/g to about 10⁹ pfu/g dry wt of animal feed. In a further example, the amount of the one or more than one strain of bacteriophage, one or more phage components from one or more than one strain of bacteriophage, or a combination thereof, may be from about 10⁴pfu/g to about 10⁸ pfu/g dry wt of animal feed. Bacteriophages that are specific to a target pathogen, but that are found outside the facility but in the same location as the facility may also be used as described above. Similarly, bacteriophage obtained from one or more libraries or collections may be used directly, or they may supplement the location-specific bacteriophage within the treatment described above.

The present invention can be used for animal feed or liquid animal feed destined for any type of animal, including but not limited to livestock, poultry, domestic, or aquaculture. For example, but without wishing to be limiting in any manner, the treated animal feed, or treated liquid animal feed, made according to the present invention, may be used for feeding swine, poultry, beef, and other livestock such as goats, sheep etc., as well as animals in stables, within petting zoos or other animal rearing systems, kennel systems, and aquaculture systems. However, it is to be understood that the bacteriophage, phage components, or both may be administered to an animal via other routes including but not limited to orally, inhalation, injection, intramuscular, intraperitonial, intrathecal, vaginal, rectal, topical or a combination thereof, as required.

The animal should receive the one, or more than one bacteriophages or phage components in any amount effective for reducing the population of target pathogen in the animal. For example, the bacteriophages can be administered at a dosage in the range of about 10³ to about 10¹³ pfu per animal per treatment, or any amount there between, for example, about 10³, 10⁴, 10⁵, 10⁶, 10⁷, 10⁸, 10⁹, 10¹⁰, 10¹¹, 10¹² or 10¹³ pfu per animal per treatment for the desired period of time. The animals are treated 1 to 3 times a day depending upon the indication being treated. The bacteriophages may be administered in a treatment dosage of about 10³ to about 10¹³, per day for a period of 1 to about 10 days, or any amount there between, for example 1, 2, 3, 4, 5, 6, 7, 8, 9, or 10 days prior to further processing of the food animal. In the case of treating a disease in food animals or other farm animals, the treatment is provided 1 to 3 times a day depending upon the indication or for 1 to about 10 days or any amount there between, for example 1, 2, 3, 4, 5, 6, 7, 8, 9, or 10 days. If a maintenance dose is desired for the food animal, the phage preparation is administered at about 10² to about 10¹¹ per day. For example, the maintenance dose may be about 10², 10³, 10⁴, 10⁵, 10⁶, 10⁷, 10⁸ or 10⁹, 10¹⁰, 10¹¹ pfu per day for a desired period of time, for example but not limited to about 10 to about 180 days, about 20 to about 90 days, or about 20 to about 60 days. The duration of administering the maintenance dose depends upon the food animal being treated with the shortest time for poultry and the most extended time for cattle. Alternatively, the administration of bacteriophages or phage components may be done in a treatment dosage of about 10⁷ to about 10¹¹ per day for a period of 1 to about 10 days, or any amount there between, for example 1, 2, 3, 4, 5, 6, 7, 8, 9, or 10 days, followed by a maintenance dosage of about 10³ to about 10⁸ pfu for a desired period of time, for example but not limited to about 10 to about 180 days, about 30 to about 90 days, or about 30 to about 60 days, prior to further processing of the animal.

Therefore, the present invention provides a method for reducing a population of one or more than one target pathogen in an animal in an animal rearing facility comprising, administering one or more than one bacteriophage strain isolated from the environment of the facility or the geographic region in which the facility is located or phage component derived from such phages, or both, to the animal, such that the one, or more than one bacteriophage strain adsorbs to the one, or more than one target pathogen, thereby reducing the one, or more than one pathogen from the animal. Furthermore, in the step of administration, the one or more than one bacteriophage strain or phage component, or both may for example, be administered to the animal for a period of about 1 to about 10 days, or from about 3 to about 7 days, after which time, in the case of food animals, the animal may be retained in the animal rearing facility, or aquatic enclosure, until it is ready for slaughter or in the case of slaughter ready animals sent for slaughter. In this case, as a result of the treatment period, the pathogen load in the animals is reduced by a minimum of 1-2 logs. This method not only helps in treatment and prevention of disease in growing animals but also ensures that animals going to slaughter comprise a reduced pathogen load and that cleaner animals are being processed within the processing plant. This also helps reduce the load of the target pathogenic bacteria in the environment of the animal rearing facility over time. In the case of other domesticated animals such as horses, dogs, cats and others, the animals are maintained in their normal habitat. The treatments help clear the bacterial infection in the animals and thus clear the animal of the disease.

The present invention further provides a method for isolating highly efficacious bacteriophages from the environment of the facility where the treatment is to be carried out or the site, local area, region, or continent in which the facility is located, and reducing a population of one, or more than one target pathogen present in an animal, comprising, administering one or more than one location-specific bacteriophage strain isolated from the site, local area, region, or continent of the facility in which the facility is located or phage component derived from these phages, or both, to the animal at a dosage from about 10³ to about 10¹³ pfu per animal per treatment for a desired period of time, such that the one or more than one bacteriophage strain, or phage components, acts to clear the one or more than one pathogen from the animal.

By isolating bacteriophages having a high degree of specificity towards isolates of the target pathogen from the environment of the facility where the animals are being treated or the geographic region in which the facility is located, provides a very efficacious system for reducing the pathogen load in animals as well as treating the animals at animal rearing facilities. Using this invention a highly effective all-natural treatment tailored to the facility can be developed, no new chemicals are introduced to the environment of the facility, target pathogen eliminated in a highly selective manner, reduce incidence of antibiotic resistance development and treatment for any new variant of the target pathogen that is observed can be quickly developed and the bio-environment in the facility is maintained. All this also helps in achieving improved and environmentally friendly treatment options for the animals and increased safety of our food supply.

Therefore, the present invention also provides a method for reducing a population of one, or more than one target pathogen present within an animal rearing facility, for example but not limited to a feedlot, a rearing enclosure, for example a barn or pen, a stable, a petting zoo, an aquaculture facility and the like. The method comprising, isolating bacteriophages highly specific to the target pathogens in the facility administering one or more than one controlled release bacteriophage strain, or phage components that are capable of adsorbing to and killing the target pathogen, to animal feed, drinking water, provided in any other edible format, or a combination thereof, such that the one or more than one controlled release bacteriophage strain or phage component, or both, is released within the feed, the drinking water, a digestive tract of the animal, at the location of its application in the animals, in manure, or a combination thereof, and reduces the population of the one or more than one target pathogen within the animal holding facility.

The present invention also provides a method for reducing bacterial diseases within aquaculture systems involved in the production of fish, shrimp, crab and other aquatic animals. In this method, bacteriophages are isolated from aquatic facilities in a given region using similar protocols as described above. The isolated bacteriophage may be stored within a collection or library as noted above for use in treatment regimes in other aquatic facilities in the region. Treatments with region-targeted bacteriophage or phage components are administered by incorporating phage in animal feed, by adding liquid phage to water, by delivering phage to the sediment by immobilizing and encapsulating phage and allowing it to reach the sediment in an appropriate precipitate form for example using a coagulating agent to form an aggregate or colloidal gel, flocculation, for example using a clarifying agent such as alum, (hydrated potassium aluminum sulfate), by coating microscopic beads (such as sephadex beads, carbohydrate coated beads, polystyrene beads, polymeric beads, plastic beads, or encapsulated phage), for example having a diameter from about 0.1-100 μm or any size there between, for example 10 μm, with phages and adding them to water which will help deliver phages to the animals through different routes including gills, by injecting either intramuscular, intraperitonial, intrathecal, by administering topically, or a combination of these methods. The amount of phage or phage components may vary depending upon the treatment regime used, for example 10³-10¹³ pfu/gm feed, or any amount therebetween, or 10³-10¹³ pfu/gm microscopic bead, coagulant, flocculent, or any amount therebetween, or 10³-10¹³ pfu/ml for example, for aquatic treatment regimes. These treatments may be repeated as required, for example every 6, 12, 18, 24, 30, 36, 42, 48, 60, 72, 94, hours, or any time there between, or from 1, 2, 3, 4, 5, 6, 7, 8, 9, 10 days or any time period therebetween. Bacteriophages that are specific to a target aquatic pathogen, but that are found outside the facility but in the same location as the aquatic facility may also be used as described above. Similarly, bacteriophage obtained from one or more libraries or collections may be used directly, or they may supplement the location-specific bacteriophage, within the treatment described above.

The present invention will be further illustrated in the following examples.

### EXAMPLES

### Example 1

Isolation of Causative Pathogen from a Farm (Site) Local Area, or Region

Causative pathogens (target pathogens) that cause disease in animals as well as those that are important in food safety are targeted. Swabs from infected areas of farm animals as well as manure and water samples are collected from different parts of the animal rearing facilities. Samples are also collected from several such facilities in the geographic region of the facility. Soil and water samples from the vicinity of the animal rearing facility are also collected for analysis. These samples are used for isolating the causative bacterial pathogen using standard bacteriology protocols (e.g. Diagnostic Bacteriology Protocols, (1995), J. Howard and D. M. Whitcombe; which is incorporated herein by reference). The isolated bacteria are characterized and the species and serotypes determined using standard microbiological and molecular biology protocols (Sambrook et al., 1989, Molecular cloning: a laboratory manual, Cold Spring Harbor Laboratory, Cold Spring Harbor, N.Y.; Diagnostic Bacteriology Protocols, (1995), J. Howard and D. M. Whitcombe which are incorporated herein by reference). These bacteria are placed in a collection for use in the development of bacteriophage-based treatment and represent the targeted bacterial population for each facility. The most prevalent strains are pooled together and used as the target bacterial panel for bacteriophage isolation.

### Example 2

### Isolation of Bacteriophages and Establishing Farm Specific Distribution

For bacteriophage isolation, samples from several areas in a rearing facility including swabs from a cross section of animals which include healthy animals, those showing signs of disease, those showing signs of recovery etc., manure, water from water troughs and other water bodies in the facility, animal bedding, animal handling machinery and tools and other areas in the facility are collected. Samples are also collected from several such facilities in the geographic region of the facility. Soil and water samples from the general vicinity of the animal rearing facilities are also collected for bacteriophage isolation. Samples (swabs, manure, bedding, water etc.) are taken in appropriate media and all water soluble material extracted. An aliquot of the extract is incubated with the target bacterial panel for that region as obtained using the method described in Example 1, and plated onto suitable agar plates prepared in appropriate selective media. Plates are incubated overnight at the appropriate temperature and any phage plaques observed are isolated and purified as per standard phage purification protocols (Sambrook et al (1989) Molecular cloning: a laboratory manual, Cold Spring Harbor Laboratory, Cold Spring Harbor, N.Y.). Isolated phages are further purified by repeated plating and used for further characterization. Bacteriophages purified as outlined above are initially plated on individual bacterial isolates in the panel used for bacteriophage isolation and the one on which they plate most efficiently identified and used for subsequent propagation. Using this protocol, a bank of bacteriophages specific to each facility is prepared as well as a distribution of phages in the geographic region of the facility is established. From this collection, phages that have the broadest host range against pathogens isolated from different facilities in the region and are the most efficacious against these bacterial isolates are selected to prepare a treatment phage panel for this geographic region.

### Example 3

### Bacteriophage Amplification and Titration

Purified phages isolated as outlined above in Example 2, are amplified using the strain of the pathogen on which they plate most efficiently. Purified phage and bacteria are mixed together, let stand at room temperature for 10 minutes, and amplified according to standard protocols commonly used in the art (Sambrook et al (1989) Molecular cloning: a laboratory manual, Cold Spring Harbor Laboratory, Cold Spring Harbor, N.Y.). Amplified samples in selective broth are filter sterilized and stored at 4° C. until use.

Concentration of bacteriophage solutions are determined using standard phage titration protocols (Sambrook et al (1989) Molecular cloning: a laboratory manual, Cold Spring Harbor Laboratory, Cold Spring Harbor, N.Y.). Preparations containing phages are diluted with suitable media, mixed and incubated with the pathogen of interest for 10 minutes and plated onto agar plates prepared in appropriate selective media. The concentration of phages is determined from the number of plaques obtained at different dilutions and multiplying with the appropriate dilution factor.

### Example 4

### Bacteriophage Characterization and Selection for Treatment

Bacteriophages most efficacious against key isolates of the target pathogen identified at the animal rearing facility, as determined by their efficiency of plating on some of the key bacterial isolates, are characterized further. The efficiency of plating on each of the most prevalent bacterial isolates in the facility is first established to confirm their host range and coverage. Those showing a broad host range and good plating efficacy are characterized further. Properties of the phages studied include molecular characterization such as RFLP profile using multiple enzymes, assays for transduction potential, sequence of their genomes to confirm the lack of undesirable elements such as virulence factors, toxins and others. Bacteriophages that show a good safety profile and are distinct from each other at the molecular level are then categorized based on their plating efficiency against the different isolates of the target pathogen to generate a Treatment Phage Bank for the target pathogen in that region. Phages selected for treatment are purified using protocols known in the art for phage purification. Phages from this collection are used as needed to develop the phage cocktail required for treatment.

### Example 5

### Preparation of Bacteriophage for Treatment

Bacteriophages selected for treatment, or phage components prepared from these phages are administered to the animals by any one of the following methods: liquid phage sprayed onto the animal feed; liquid phage added to water, lyophilized phage preparation applied onto feed; phage immobilized onto a solid support by passive adsorption and added to feed; liquid phage covalently bound to beads and mixed-in with feed; phage covalently bound to beads and added to water. Alternatively, liquid phage is mixed with a liquid stabilizer/coating agent and applied onto feed after pelletization to prepare bacteriophage containing feed as outlined in Example 6. Bacteriophage containing feed can also be prepared using phage immobilized onto beads and applying it to pelletized feed. Bacteriophage containing feed is stored separately and mixed-in with regular feed at the time of application.

For other therapeutic preparations, either liquid phage, phage encapsulated using suitable encapsulation protocols or phage immobilized on beads is used. The phage preparations are mixed in with appropriate excipients such as cream, lotion, jelly or lubricant before use. Bacteriophage or phage components described above may also be administered by inhalation, or injection either intramuscular, intraperitonial, or intrathecal, or by administering rectally, topically, or a combination of these methods

### Example 6

### Preparation of Active Bacteriophage Containing Pellets for Administering to Farm Animals

Use of bacteriophage containing pelleted feed is a convenient way to administer feed to food production animals such as poultry and swine, pet animals such as dogs and cats, aquatic animals and the like. This method is also well suited for delivering phages to these animals. Immobilized, lyophilized phage preparation are admixed with the pellets immediately after the pelleting process or anytime thereafter. For this application, bacteriophages or phage components that are prepared in any of the formats (lyophilized bacteriophages or phage components, bacteriophages or phage components that are immobilized on a matrix, bacteriophages or phage components that are encapsulated, bacteriophages or phage components that are provided in capsule form, bacteriophages or phage components that are provided in tablet form, or a combination thereof) described earlier can be used. As an example, use of bacteriophages immobilized covalently onto a solid support is presented here. Bacteriophages are immobilized covalently onto a solid support using technology as outlined in U.S. Pat. No. 7,482,115, mixed-in with liquid binders such as molasses, desugared molasses, sugar syrup, corn steep liquor, condensed liquid whey, edible oil, wax, edible polymers, gums, vegetable gums, cellulose, or other liquid binders known to one of skill in the art, that are suitable for spraying and that have the property of being sticky, and applied to the pellets as they are being extruded from the pelletizer and cooled to temperatures below 50° C. This process can be easily achieved by minor modification to pelleting machines that are currently available on the market. Once cooled to room temperature, the bacteriophage containing pellets are stored separately and used. The pelleted feed can also be sprayed with phage or phage components after the pelleting process is completed. These bacteriophage containing pellets can be mixed-in with regular pelleted feed at a defined ratio for administration to the animals.

### Example 7

### Treatment of Farm Animals to Improve Animal Health

Bacterial diseases causing production issues can be addressed using bacteriophages targeted to the farm in question. Treatments with bacteriophage or phage components may be administered by adding to animal feed or drinking water, by inhalation, or injection either intramuscular, intraperitonial, or intrathecal, or by administering rectally, topically, or a combination of these methods. As an example, treatment of post weaning diarrhea in piglets by applying the treatment in feed is presented here.

Post weaning diarrhea is a bacterial disease in weaned piglets caused primarily by E. coli K88 bacteria. This infection leads to production issues and in some severe cases, dehydration and death. One major issue with this disease is that the pathogen is becoming multi drug resistant and treatment options are becoming limited. The approach presented in this invention is ideal for treating this infection.

As a first step in the process, manure is collected from 50 piglets with post weaning diarrhea in the facility and the target pathogen E. coli K88 isolated. The relative abundance and level of E. coli K88 in the samples is determined. E. coli K88 specific phages from the Phage Bank prepared using samples from facilities in this region are then tested on isolates from this herd and the most efficacious phages selected. The selected phages covalently immobilized on to microbeads (polymeric beads) are used for this application. Other methods of phage preparation such as non-covalent immobilization, lyophilization, encapsulation, liquid phage, phages in a capsule or a tablet format etc. can also be used as needed.

Bacteriophage containing pelleted feed is mixed in with regular fed so as to provide a final bacteriophage concentration of from 10⁷ to 10¹⁰ pfu/animal/application. In this example, a concentration of 10⁹ pfu/animal/application is used. The amount of treated feed provided is such that each animal gets the required dose over a span of 6-12 hrs. A single application is given each day. Once the animals consume all the bacteriophage containing feed, regular pelleted feed is provided ad-libitum. This process is continued for 7-10 days. Clinical signs of all the animals including score for severity of the diarrhea are monitored throughout the study. At the end of the treatment period, fecal samples are taken from the animals that were tested earlier and the level of E. coli K88 determined.

Using phages highly specific to the E. coli K88 isolates found in the facility, the treatment is highly efficacious with many of the animals showing significant clinical improvement. The bacterial level is also reduced by a minimum of 1-2 logs. If any further improvements in the level of reduction in pathogen levels are needed, adjustments to the treatment protocol, such as modifications to the amount of phage used, the mode and duration of treatment etc., are made.

Alternate treatment methods such as using encapsulated phages, phages immobilized on a support soluble in water, phages covalently immobilized onto a support such as micro beads etc. may also be used. In addition, the phages may be presented in water, as an aerosol, or other formats. Slight protocol modification may be necessary for each of these applications.

### Example 8

### Treatment of Production Animals to Improve Food Safety

Treatment of broiler chicken to eliminate Salmonella Enteritidis is presented here as a non-limiting example of the use of the present method. The treatment protocols for the different target production animals (cattle, swine etc.) and pathogens (E. coli O157:H7, Campylobacter jejuni etc.) may need to be modified to suit the different animal systems, however, the general concept outlined within this invention remain the same. The treatment is designed to use bacteriophages from a region-specific treatment phage bank prepared earlier.

A flock of 100 broiler chicken positive for Salmonella Enteritidis and 2 weeks from slaughter is selected for this treatment. Fecal and cloacal samples are collected from all chicken and the distribution and level of the Salmonella Enteritidis serotype is determined. Salmonella Enteritidis phages from the Phage Bank prepared using samples from this region are then tested on the isolates obtained from this flock and the most efficacious phages for this application are selected. The presence and level of these or similar phages in the facility is also determined. The selected phages that covalently immobilized on beads are used in this application. Other methods of phage preparation such as non-covalent immobilization, lyophilization, encapsulation etc. can also be used as needed.

Treatment of these birds is started 1 week before slaughter. Prior to application of the bacteriophage containing pelleted feed to the flock, all feed is removed from the birds. Bacteriophage containing pelleted feed prepared as outlined earlier is mixed-in with regular pelleted poultry feed to provide a final concentration of from 10⁷ to 10¹⁰ pfu/bird/application. In this example a phage concentration of 10⁸ pfu/bird/application is used. The amount of treated feed provided is such that each bird gets the required dose in a span of 6-12hrs. A single application is given each day. Once the birds have eaten all the bacteriophage containing feed, regular pelleted poultry feed is provided ad-libitum. This process is repeated for 5-7 days at the end of which fecal samples and cloacal swabs are taken again from 100 birds and the level of Salmonella Enteritidis is determined.

Using this highly specific treatment approach, Salmonella Enteritidis levels in the flock and also in the facility can be reduced by a minimum of 1-2 logs. If any further improvements in the level of reduction in pathogen levels are needed, adjustments to the treatment protocol, such as modifications to the amount of phage used, the mode and duration of treatment etc., are made.

Alternate treatment methods such as using encapsulated phages, phages immobilized on a support soluble in water, phages non-covalently immobilized onto a solid support etc. may also be used. In addition, the phages may be presented in water, as an aerosol, or in other formats. Slight protocol modification may be necessary for these alternate modes of applications.

### Example 9

### Treatment of Bacterial Pathogen in an Aquatic Facility Using Phages

Bacterial diseases causing production issues in aquaculture (fish, shrimp, crab and other aquatic animals) can be addressed using bacteriophages targeted to aquatic facilities in a given region. Treatments with bacteriophage or phage components may be administered by incorporating phage in animal feed, or by adding liquid phage to water, or by delivering phage to the sediment by immobilizing and encapsulating phage and allowing it to reach the sediment in an appropriate precipitate form, or by coating microscopic beads with phages and adding them to water which will help deliver phages to the animals through different routes including gills, or by injecting either intramuscular, intraperitonial, or intrathecal, or by administering topically, or a combination of these methods. As an example, treatment of Aeromonas salmonicida infection in fish by applying the treatment in feed is presented here.

Water and sediment from a given aquatic facility are collected and used for isolating the bacterial pathogen (Aeromonas salmonicida in this non-limiting example) using standard bacteriology protocols and characterized as outlined earlier (Example 1). The isolated bacteria is placed in a collection and used for isolating/identifying the appropriate phage product to be used for this application.

For phage isolation, water (1 L to 100 L depending on phage concentration in the water) and sediment (0.1 L to 10 L depending on phage concentration in the sediment) is collected from different aquatic facilities in a given region. The collected water is concentrated by precipitation, for example using tangential flow filtration, hollow fiber systems, other commercially available concentration systems or other concentration methods as are known to one of skill in the art. In the case of sediment, water will be added to the sediment, mixed well and water soluble extract used for phage isolation. Phage are isolated using protocols outlined earlier (Example 2). Isolated phages are added to the phage library and used as needed.

As a first step in the treatment process, field isolates of Aeromonas salmonicida are isolated from water and sediment samples from the aquaculture facility. Appropriate phages required to treat this infection are identified from the phage library prepared from samples taken from the region as outlined earlier. Phages are then applied to feed by the method outlined in earlier examples (Example 5 and 6) or by other methods as are known in the art, mixed in with normal feed and fed to the animals being treated in the aquatic facility.

Selected phage preparations are applied to the feed at a concentration of 10³ to 10¹³ pfu/gram of feed. In this example a phage concentration of 10⁹ pfu/gram of feed is used. Without wishing to be bound by theory, once the feed is consumed by the animals, phages are released in the digestive tract and the target pathogenic bacteria present in the gut will be eliminated. Phages released during this process will also be excreted into the water and will help reduce local target pathogenic bacterial population. The treatment is administered to the affected facilities for a period of 3-10 days, or any time period therebetween. The water samples are tested for the presence of the Aeromonas salmonicida to confirm effectiveness of the treatment. Using this treatment protocol, the concentration of the target pathogenic bacteria will be reduced by a minimum of 1-2 logs. If any further improvements in the level of reduction in pathogen levels are needed, adjustments to the treatment protocol, such as modifications to the amount of phage used, the mode and duration of treatment etc., can be made.

All citations are hereby incorporated by reference.

The present invention has been described with regard to one or more embodiments. However, it will be apparent to persons skilled in the art that a number of variations and modifications can be made without departing from the scope of the invention as defined in the claims

1. A method for reducing a population of one or more than one pathogenic bacteria in an animal being reared in an animal rearing facility comprising,
using one or more than one naturally-occurring bacteriophages isolated from or found within 0-3000 kms from the animal rearing facility and selected to be highly specific to the one or more than one pathogenic bacteria present in the facility, a phage component derived from the one or more than one naturally-occurring bacteriophages, or a combination thereof, to produce one or more than one location-specific bacteriophage preparation, and
administering the one or more than one location-specific bacteriophage preparation to the animal, thereby reducing the population of the one or more than one pathogenic bacteria.

2. The method of claim 1, wherein the one or more than one naturally-occurring bacteriophages are isolated from or found in the animal rearing facility or the surrounding environment of the animal rearing facility.

3. The method of claim 1, wherein the one or more than one naturally-occurring bacteriophages found in the animal rearing facility or the surrounding region are selected from a collection of bacteriophages isolated from different facilities either in the same site, local area, region, continent, or a different site, local area, region, continent, or a global phage collection or a combination thereof.

4. The method of claim 1, wherein each of the one or more than one naturally-occurring bacteriophages are distinguishable from each other by RFLP, host range using multiple bacterial isolates of the one or more than one pathogenic bacteria.

5. The method of claim 1, wherein the phage component is selected from the group consisting of a phage tail, phage protein, and a combination thereof.

6. The method of claim 1, wherein in the step of administering, the one or more than one location-specific bacteriophage preparation is administered in a stabilized liquid form, in an immobilized and lyophilized form, in an encapsulated form, in a tablet form, in a capsule form, immobilized onto a solid support form, in a cream, in a lotion, in a gel or in a lubricant, or a combination thereof.

7. (canceled)

8. The method of claim 1, wherein in the step of administering, the one or more than one location-specific bacteriophage preparation is administered in water, admixed with liquid feed, admixed with pelleted feed, top-dressed onto feed, or a combination thereof.

9. The method of claim 1, wherein in the step of administering, the one or more than one location-specific bacteriophage preparation is administered orally, by inhalation, by injection, intraperitonially, intrathecally, rectally, topically or a combination thereof.

10. The method of claim 1, wherein in the step of administering, the one or more than one location-specific bacteriophage preparation is applied at a rate of 1 to 3 treatments per day at a treatment dose of about 10³ to about 10¹³ pfu per animal per treatment, with a treatment duration from about 1 to about 15 days.

11. (canceled)

12. The method of claim 10, wherein, the one or more than one location-specific bacteriophage preparation is administered at a maintenance dose of about 10³ to about 10¹⁰ pfu per animal per treatment.

13. The method of claim 10, wherein, in the step of administering, the one or more than one location-specific bacteriophage preparation is administered at a treatment dose of about 10³ to about 10¹³ pfu per animal per treatment, followed by a maintenance dose of about 10³ to about 10¹⁰ pfu per animal per treatment.

14. The method of claim 1, wherein the animal is selected from the group consisting of a food animal and a non-food animal,
wherein the food animal is selected from group consisting of beef cattle, poultry, swine, sheep, fish, shrimp, and crab.

15. (canceled)

16. A method of preparing bacteriophage containing pelleted feed comprising, adding the one or more than one location-specific bacteriophage preparation of claim 1 to a liquid binder or other media to produce a mixed preparation, coating pelletized feed after extrusion with the mixed preparation to produce a coated pelletized feed, and cooling the coated pelletized feed to below 50° C.

17. The method of claim 16 wherein the liquid binder is selected from the group consisting of molasses, desugared molasses, sugar syrup, corn steep liquor, condensed liquid whey, edible oil, wax, edible polymers, gums, vegetable gums, and cellulose.

18. The method of claim 16, wherein the one or more than one location-specific bacteriophage preparation is in stabilized liquid form, immobilized and lyophilized, encapsulated, immobilized onto a solid support or a combination thereof prior to being added to the liquid binder or other media.

19. The method of claim 16 wherein the one or more than one pathogenic bacteria is selected from the group consisting of Campylobacter, Salmonella, E.coli O157:H7, Escherichia coli, Streptococcus spp., Staphylococcus aureus, Treponema, Clostridium spp, Aeromonas, Renibacterium, Edwardsiella, Vibrio, and other pathogens involved in food safety or causing disease in animals.

20-22. (canceled)

23. The method of claim 16, wherein the animal being treated is a food animal selected from the group consisting of beef cattle, poultry, swine, sheep, fish, shrimp, and crab, so as to improve health of the food animal, and to improve safety of meat and meat products derived from the food animal.

24-25. (canceled)

26. A method of reducing pathogenic bacteria within one or more than one animal being reared in an animal rearing facility comprising,
a. identifying location-specific isolates of one or more than one pathogenic bacteria at or near the animal rearing facility;
b. identifying one or more than one bacteriophage strain naturally present in the animal rearing facility or site, local area, region, or continent, of the animal rearing facility, the one or more than one bacteriophage strain exhibits antibacterial activity against the one or more pathogenic bacteria, to obtain a location-specific bacteriophage preparation, the location-specific bacteriophage preparation comprising the one or more than one bacteriophage strain, phage components obtained from the one or more than one bacteriophage strain, or a combination thereof, wherein the one or more than one bacteriophage strain naturally present in the animal rearing facility is selected from a bacteriophage collection comprising bacteriophages isolated from:
i) one or more different animal rearing facilities located at the site, the local area, the region, the continent,
ii) one or more different animal rearing facilities located at a different site, a different local area, a different region, a different continent,
iii) a global phage collection, or
iii) any combination of i), ii), and iii);
c. inoculating the one or more than one animal with the location-specific bacteriophage preparation, thereby reducing the pathogenic bacteria within the one or more than one animal being reared in the animal rearing facility.

27. The method of claim 26 comprising an step (d) of repeating steps (a) to (c) after a period of time to reduce any additional pathogenic bacteria that may be identified within the rearing facility.

28. The method of claim 28, wherein the period of time is a time interval of from about 1 month to about 48 months or any time interval therebetween.
